(19) United States
(12) Patent Application Publication
(10) Pub. No.: US 2013/0309234 A1
(43) Pub. Date: Nov. 21, 2013

(54) CORRELATION OF DE NOVO-INDUCED TUMOR-ASSOCIATED HUMORAL IMMUNE RESPONSES WITH IMPROVED CLINICAL OUTCOME

(71) Applicant: Trion Pharma GmbH, Munich (DE)

(72) Inventor: Horst Lindhofer, Munich (DE)

(21) Appl. No.: 13/827,965

(22) Filed: Mar. 14, 2013

Related U.S. Application Data
(60) Provisional application No. 61/649,130, filed on May 18, 2012.

(51) Int. Cl. G01N 33/68; A61K 39/395

(52) U.S. Cl. CPC G01N 33/6854; A61K 39/39558
USPC 424/136.1

(57) ABSTRACT

The present invention refers to a method for predicting an improved therapeutic benefit for an individual suffering from a tumor carrying a known tumor-associated cell surface antigen (such as EpCAM). The present invention further refers to a method for the induction of secondary humoral immune responses directed against a second tumor-associated antigen (e.g., another tumor-associated antigen, like HER2/neu) different from the first tumor-associated antigen in an individual suffering from a tumor comprising tumor cells expressing the first tumor-associated antigen, e.g., EpCAM.)

### RELATED APPLICATIONS

This application claims priority to U.S. Provisional Patent Application No. 61/649,130, filed May 18, 2012, the contents of which are hereby incorporated by reference in the entirety for all purposes.

### BACKGROUND OF THE INVENTION

1. Field of the invention

The present invention refers to a method for predicting an improved therapeutic benefit for an individual suffering from a tumor carrying a known tumor-associated cell surface antigen (such as EpCAM) and to kits to be used in order to predict said therapeutic benefit. The present invention further refers to a method for the induction of secondary humoral immune responses directed against a second tumor-associated antigen (e.g., another tumor-associated antigen, like Her2/neu) different from the first tumor-associated antigen in an individual suffering from a tumor comprising tumor cells expressing the first tumor-associated antigen, e.g., EpCAM.

2. Description of the related art

It is already known that conventional therapeutic antibodies, e.g. trastuzumab, in combination with chemotherapy led to the generation of humoral and cell-mediated immune responses that, in turn, could be correlated with a beneficial clinical outcome of breast cancer patients (Taylor et al., Clin. Cancer Res. 13: 5133, 2007). Moreover, it is suggested that the combined antitumor activity of rituximab and chemotherapy elicit analogous immune responses that might account for the high clinical response rate in patients suffering from malignant B cell diseases (Coiffier et al., Blood 116: 2040, 2010).

### BRIEF SUMMARY OF THE INVENTION

This invention is based on the surprising discovery by the present inventor that, upon treating patients suffering from a tumor that comprises tumor cells expressing a first tumor-associated cell surface antigen (such as EpCAM) with a bispecific antibody capable of binding to the first tumor-associated antigen, CD3, and Fc receptors, the effectiveness of such bispecific antibody treatment or an improved therapeutic benefit can be predicted. More specifically, in some but not all patients, humoral immunity against a second, non-targeted tumor-associated antigen (i.e., a tumor antigen other than e.g. EpCAM that is also expressed by the tumor cells, such as Her2/neu) can be induced by the administration of a bispecific antibody targeting the first tumor-associated antigen. Such secondary humoral immunity is detected by measuring and comparing the levels of antibodies against the second tumor antigen in a patient's blood (or plasma or serum) samples taken before and after receiving bispecific antibody administration. An increase in the level of antibodies against the second tumor antigen after the bispecific antibody administration but not resulted from any concurrent treatment the patient is receiving (e.g., chemotherapy) is indication of induced secondary humoral immunity, which in turn indicates improved therapeutic benefits (for example, among patients suffering from increased formation of ascites fluid, such benefit may include an increase in puncture-free survival time).

Upon recognizing whether for a particular patient such improved therapeutic benefits are available from the bispecific antibody treatment, a physician may modify future treatment plan for the patient accordingly. For example, if the improved therapeutic benefits are predicted, the bispecific antibody treatment may be used or continued; on the other hand, if no improved therapeutic benefits are predicted, the bispecific antibody treatment may be discontinued in favor of an alternative treatment plan.

According to a first aspect of the present invention, there is provided a method for predicting an improved therapeutic benefit for an individual suffering from a tumor comprising tumor cells expressing a first tumor-associated cell surface antigen, the method comprising:
(a) administering to the individual a therapeutically effective amount of a trifunctional bispecific antibody with the following properties
binding to a T cell via CD3
binding to the first tumor-associated cell surface antigen
binding via its Fc-portion to Fcy-receptor type I, II and/or III positive cells;
(b) determining the level of antibodies against a second tumor-associated cell surface antigen in a blood sample taken from the individual after step (a);
(c) comparing the level of antibodies from step (b) with the level of antibodies against the second tumor-associated antigen in a blood sample taken from the individual prior to step (a);
(d) when an increase in the level of antibodies is detected in step (c), an improved therapeutic benefit is indicated.

If upon completion of step (c) an improved therapeutic benefit or a favourable therapeutic outcome in a patient is indicated, the patient may proceed to begin the therapeutic regimen of administration of a trifunctional bispecific antibody described in (a); on the other hand, if the comparison results of step (c) indicate no improved therapeutic benefit, then the attending physician may consider giving the patient an alternative therapeutic plan for treating the tumor, other than administration of trifunctional bispecific antibody as described in (a) or additionally to the trifunctional antibody as described in (a).

According to a second aspect of the present invention, there is also provided a method for the induction of secondary humoral immune responses directed against non-targeted second tumor-associated antigens in an individual suffering from a tumor carrying a first tumor-associated antigen like EpCAM-on the surface of tumor cells comprising administering to an individual in need thereof a therapeutically effective amount of a trifunctional bispecific antibody with the following properties
binding to a T cell via CD3
binding to the first tumor-associated antigen, e.g., EpCAM or HER2/neu
binding via its Fc-portion to Fcy-receptor type I, II and/or III positive cells

inducing in said individual an augmented or de novo humoral immune response directed against at least one second tumor-associated antigen (e.g., HER2/neu or EpCAM) different from said first tumor-associated antigen (e.g., EpCAM or HER2/neu) by said administration of a trifunctional bispecific antibody, wherein said induction of a humoral immune response is independent from concurrent chemotherapeutic interventions.

According to a third aspect of the invention, there is provided a kit for predicting an improved therapeutic benefit for an individual suffering from a tumor comprising tumor cells expressing a first tumor-associated cell surface antigen, the kit comprising means capable of detecting of a second antibody raised against a second tumor-associated antigen in a biological sample of a patient treated with a trifunctional bispecific antibody directed against a first tumor-associated antigen and means for determining the amount of said second antibody in the sample and means for comparing the levels of said antibody against said second tumor-associated antigen raised before and after treatment of said patient with a trifunctional bispecific antibody directed against a first tumor-associated antigen.

According to a forth aspect of the invention, there is provided a method for the induction of secondary humoral immune responses directed against non-targeted second tumor-associated antigens in an individual suffering from a tumor carrying a first tumor-associated antigen on the surface of tumor cells comprising

administering to an individual in need thereof a therapeutically effective amount of a trifunctional bispecific antibody with the following properties
binding to a T cell via CD3
binding to the first tumor-associated antigen
binding via its Fc-portion to Fcg-receptor type I, II and/or III positive cells,

inducing in said individual an augmented or de novo humoral immune response directed against at least one second tumor-associated antigen different from said first tumor-associated antigen by said administration of a trifunctional bispecific antibody, wherein said induction of a humoral immune response is independent from concurrent chemotherapeutic interventions, and wherein the amount of said antibody induced against said second or further tumor-associated antigen after administration of said therapeutically effective amount of a trifunctional bispecific antibody is compared with the amount of said second or further antibody before administration of said therapeutically effective amount of a trifunctional bispecific antibody wherein an increase in the amount of said antibody induced against said second or further tumor-associated antigen indicates an improved therapeutic benefit.

According to a fifth aspect of the invention, there is provided a method for treating a patient suffering from a tumor expressing multiple tumor-associated antigens (e.g., a first tumor-associated antigen, a second tumor-associated antigen, optionally more), the method comprising administering to the patient a therapeutically effective amount of a trifunctional antibody with the following properties:
binding to a T cell via CD3;
binding to a tumor-associated antigen; and
binding via its Fc-portion to Fcg-receptor type I, II and/or III positive cells, wherein the patient has been previously identified by the method of claim 1 to have an improved therapeutic benefit.

### BRIEF DESCRIPTION OF THE FIGURES

The figures show with reference to the example of the invention the following:

FIG. 1. Anti-EpCAM IgG concentrations measured at screening.

FIG. 2. Prominent increase of initial anti-EpCAM immunoglobulin titers in 11 of 23 patients (48%). X-fold increase of anti-EpCAM IgG response in comparison to screening value which was set to 1. Responders show a median increase of 3-4 times starting at day io after the first catumaxomab infusion. Moreover, all analyzed patients were positive for EpCAM antigen expression of ascites tumor cells (not shown).

FIG. 3. Development of HER2/neu-specific antibodies in 14 of 23 patients (6i%). In 14 patients (61%) an anti-HER2/neu-IgG response (>12.5 ng/ml) could be detected within 38 days after the start of catumaxomab therapy. Apart from one patient who was already positive at screening the anti-HER2/neu responses developed de novo, appeared 18 days after the first catumaxomab infusion and increased at day 38.

FIG. 4. Development of HER2/neu-specific antibodies in 4 of 5 patients along a second catumaxomab treatment cycle (four 3-hour infusions of 10, 20, 50 and 150 μg catumaxomab, SECIMAS study over ii days) after MA relapse more than 60 days post first catumaxomab therapy cycle.

FIG. 5. Positive correlation of anti-HER2/neu immune responses and clinical outcome indicated by PFS of CASIMAS patients suffering from MA (p=0.044, log rank test).

FIG. 6. Positive correlation between humoral anti-tumor immune responses and a trend to improved overall survival.

FIG. 7. Changes of human anti-HER2/neu autoantibody levels in the serum of cancer patients 01-02 upon anti-HER2/neu×anti-CD3 ertumaxomab treatment

FIG. 8. Changes of human anti-EpCAM autoantibody levels in the serum of cancer patients 01-04 upon anti-HER2/neu×anti-CD3 ertumaxomab treatment

Table 1. Induction of human anti-EpCAM and/or anti-HER2/neu autoantibodies upon cancer treatment of HER2/neu-positive carcinomas with the trifunctional anti-HER2/neu×anti-CD3 antibody ertumaxomab along the phase I/II ERTUSO trial

### DETAILED DESCRIPTION OF THE INVENTION

Trifunctional bispecific antibodies binding (i) to the T cell receptor complex of a T cell, (ii) to the surface-exposed tumor-associated antigen on a tumor cell and to the activating Fc-gamma receptor I, II or III on an accessory cell (i.e., natural killer cell, macrophage, monocyte, dendritic cell) have been described in U.S. Pat. No. 6,551,592 as having the capacity to induce anti-tumor immunity. The anti-tumor immunity is generated by administering an efficient amount of a trifunctional intact bispecific antibody having the following properties and effects of: (a) binding to a T cell and mediating a first activation signal thereto; (b) binding to tumor-associated antigens on a tumor cell; (c) binding, through its Fc portion (in the case of bispecific antibodies) to the Fc-gamma receptor of Fc-gamma receptor-positive cells; (d) activation of the Fc receptor-positive cell by binding to the Fc-gamma receptor-positive cell and, thereby initiating or increasing the expression of cytokines and/or co-stimulatory antigens; (e) transfer of at least one second activation signal required for physiological activation of the T cell to the T cell by the co-stimulatory antigens and/or cytokines, wherein said activation causes an up-regulation of activation markers, killing of the tumor cell, and/or T cell proliferation.

This invention describes for the first time that trAbs induce humoral antitumor responses that are beneficial for the clinical outcome of cancer patients suffering e.g. from EpCAM-positive tumors and e.g. an increased formation of ascites fluid. In contrast to conventional therapeutic antibodies like trastuzumab or rituximab, the induction of these vaccination-like effects by trAbs does not depend on chemotherapeutic co-measures.

The inventors have found that the trifunctional bispecific antibodies have the capacity to induce antigen-specific humoral immune responses against non-targeted tumor-associated antigens.

The recruitment of immune cells is accompanied by cellular activation events elicited by anti-CD3, as well as Fc-gamma-receptor engagement of trAbs supported by a proinflammatory Thi-biased cytokine milieu.

All necessary immunological factors required for long-term vaccination-like effects are stimulated along trAb-mediated therapeutic interventions. Thus, the concerted interplay of antibody-dependent cellular cytotoxicity plus the polyclonal T-cell cytotoxicity and Fc-gamma-receptor-driven induction of long-lasting immune responses after the initial tumor cell elimination represent the major hallmarks of trAb-mediated treatment of malignant diseases.

Due to all of these functional binding properties trAbs are capable of linking innate with adaptive immunity. Thus, in terms of therapeutic cancer vaccination trAbs represent immuno-modulating entities that trigger adaptive humoral and/or cell-mediated immune responses.

In other words, trAbs resemble in combination with immune cells and dying tumor cells a particular form of therapeutic cancer vaccination.

Usually, therapeutic cancer vaccines are biological preparations that improve immunity to a malignant disease. A therapeutic cancer vaccine typically contains an agent that is derived from a cancer cell. This agent stimulates the body's immune system to recognize the agent as foreign, destroy it, and remember it, so that the immune system can more rapidly recognize and destroy any of these specific tumor cells that it encounters in diseased individuals. It is absolutely required that cancer vaccines have to break already existing immune tolerance.

This invention demonstrates that administration of a trAb with the characteristics of present claim 1 and as described herein having the properties of an immuno-modulating drug with CD3-engagement in a therapeutically effective amount promotes the de novo induction of systemic secondary tumor-specific antibody responses; these responses are directed against a second tumor-associated cell surface antigen as tumor surface marker, which in turn significantly leads to the beneficial clinical treatment outcome against tumors characterized by their expression of a first tumor-associated cell surface antigen as tumor surface marker. Said first antigen might be EpCAM, said second antigen HER2/neu and said beneficial clinical result might be e.g. a longer puncture-free survival time (PFS) for removing excess ascites fluid during malignant ascites (MA). One example for the antibody is catumaxomab as described herein.

Briefly, in 61% patients (14/23) a considerable anti-HER2/neu-IgG response (>12.5 ng/ml) could be detected within 38 days after the start of catumaxomab therapy. Most importantly, more than 92% of these anti-HER2/neu antibody-positive MA patients showed de novo generation of these tumor-specific humoral immune responses indicating long-term vaccination-like effects evoked by e.g. catumaxomab treatment. Furthermore, the first immunological analysis of the ERTUSO trial revealed that upon ertumaxomab treatment of HER2/neu-positive carcinomas serum samples showed de novo induction of human anti-HER2/neu- and anti-EpCAM-specific immune responses in certain cancer patients. Thus, the humoral immune responses were not restricted to the antibody target, for instance EpCAM or HER2/neu only but also antigen spreading to non-targeted tumor-associated antigens like HER2/neu or EpCAM tumor-associated antigen occurred. Furthermore, the early onset of de novo generated tumor-specific anti-HER2/neu antibody responses in combination with beneficial PFS represents an important biomarker for the treatment outcome against EpCAM- or Her2/neu positive cancers.

For purposes of the present invention, the term “humoral immune response” refers to an IgG immune response mediated by antibody molecules, while a “cellular immune response” or “cell-mediated immune response” is one mediated by T-lymphocytes and/or other white blood cells.

The term “puncture free survival time” refers to the time period a patient suffering from malignant ascites and involving an increased formation of ascites fluid is absent of formation of new excess ascites fluid for a time period which is significantly longer than in those patients which do not have received a treatment with said trAb.

The phrase “specifically binds,” when used in the context of describing the interaction between a protein or peptide and another agent or compound (e.g., an antibody), refers to a binding reaction that is determinative of the presence of the protein in a heterogeneous population of proteins and other biologics. Thus, under designated assay conditions, the specified binding agent (e.g., an antibody) binds to a particular protein at least two times the background and does not substantially bind in a significant amount to other proteins present in the sample. Specific binding to an antibody under such conditions may require an antibody that is selected for its specificity for a particular protein or a protein but not its similar “sister” proteins. A variety of immunoassay formats may be used to select antibodies specifically immunoreactive with a particular protein or in a particular form. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective binding reaction will be at least twice background signal or noise and more typically more than 10 to 100 times background.

“An amount sufficient (or effective)” or “an effective amount” or a “therapeutically effective amount” is that amount of a therapeutic agent at which the agent exhibits its activity for the intended purpose of its administration. In therapeutic applications, an amount adequate to accomplish this is defined as the “therapeutically effective dose.” For example, an effective amount for a therapeutic agent for the purpose of treating a particular condition is an amount that when administered to a patient suffering from or at risk of developing the condition, the agent is capable of reducing or substantially eliminating the clinical symptoms of the condition or delaying or avoiding the onset of the condition.

As used in this application, an “increase” or a “decrease” refers to a detectable positive or negative change in quantity from a comparison basis, e.g., an established baseline value of the level of an antibody against a specific antigen. An increase is a positive change that is typically at least 10%, or at least 20%, or 5o%, or 100%, and can be as high as at least 2-fold or at least 5-fold or even 10-fold of the control value (i.e, the level of antibody prior to bispecific antibody administration). Similarly, a decrease is a negative change that is typically at least 10%, or at least 20%, 30%, or 5o%, or even as high as at least 80% or 90% of the control value. Other terms indicating quantitative changes or differences from a comparative basis, such as “more,” “less,” “higher,” and “lower,” are used in this application in the same fashion as described above. In contrast, the term “substantially the same” or “substantially lack of change” indicates little to no change in quantity from the control value, typically within ±10% of the standard control, or within ±5%, 2%, or even less variation from the standard control.

The inventor has found that trifunctional bispecific antibodies binding to a T-cell via CD3, binding to a first tumor-associated cell surface antigen and binding via its Fc-portion to Fc-gamma-receptor type I, II and/or III positive cells and administered to a patient suffering from a tumor expressing a first tumor-associated cell surface antigen can be used to indicate an improved therapeutical benefit if the induction of an antibody response against a second tumor-associated antigen (i.e., a humoral immune response) is determined. When for instance the trifunctional bispecific antibody is directed against EpCAM or HER2/neu as first tumor-associated antigen, induction of antibodies (secondary humoral immune responses) against at least a second tumor associated cell surface antigen (e.g., anti-HER2/neu or EpCAM) different from the first was found.

This increase in level of antibody against the second tumor-associated cell surface antigen could be correlated with a beneficial therapeutic effect. The antibody against at least a second tumor associated antigen different from the first antigen EpCAM was for instance at least anti-Her2/neu. If the first antibody was directed against Her2/neu tumor-associated antigen, the antibody triggered against a second tumor associated antigen different from the first was for instance anti-EpCAM.

When the trAb was directed against EpCAM as first tumor-associated antigen and induction of anti-HER2/neu antibodies as a secondary humoral immune response could be detected, the inventors could predict an increased puncture-free survival time in a patient suffering from an increased formation of ascites fluid as a major therapeutic advantage. This was done by correlating an increased level of antibodies against the second tumor-associated antigen with an increased ascites fluid puncture-free survival time as improved therapeutic outcome.

The antibodies to be used in the present invention will be described in detail later on. Said trifunctional bispecific antibodies are administered to an individual in need thereof in a therapeutically effective amount. Said individual suffers from a tumor and carries on a tumor cell a tumor-associated cell surface antigen, e.g., at least EpCAM. EpCAM is typically associated with adenocarcinomas. Malignant ascites may be caused by a plurality of primary tumors such as breast cancer, or ovarian carcinoma or gastrointestinal carcinomas. An increased formation of ascites fluid is typically correlated with a progressive tumor disease. The present options for a therapy of ascites include puncture, local chemotherapy, diuretic treatments or treatment with the trifunctional bispecific antibody catumaxomab. However, it has not been known up to now that trifunctional bispecific antibodies as described herein like catumaxomab (binding to a T-cell via CD3, binding to the tumor-associated antigen EpCAM and binding via its Fc-portion to Fc-gamma receptor positive cells) or ertumaxomab (binding to a T-cell via CD3, binding to the tumor-associated antigen HERR2/neu and binding via its Fc-portion to Fc-gamma receptor positive cells) are feasible of inducing secondary humoral immune responses directed to antigens which are not targeted by the trifunctional antibody applied to the patient. This is particularly true as the humoral immune responses initiated against e.g. non-EpCAM or non-HER2/neu antigens are induced even in the absence of concurrent chemotherapeutic interventions.

The antibodies for use in the invention may be administered orally in any acceptable dosage form such as capsules, tablets, aqueous suspensions, solutions or the like. The antibodies and derivatives thereof may particularly be administered parenterally. That is via the following routes of administration: subcutaneous, intravenous, intraperitoneal, intramuscular, intra-articular, intra-synovial, intrasternal, intranasal, topical, intrathecal, intrahepatic, intratumoral, intralesional, and intracranial injection or infusion techniques. Intraperitoneal application and intravenous administration are particularly preferred. Generally, the antibodies will be provided as an intravenous or intraperitoneal injection or infusion.

The antibodies of the invention may be administered alone or preferably with a pharmaceutically acceptable carrier, including acceptable adjuvants, vehicles, and excipients. All of these are familiar for those skilled in the art. One preferred carrier is physiological saline.

The effective dosage will depend on a variety of factors and it is well within the purview of a skilled physician to adjust the dosage for a given patient according to various parameters such as body weight, the goal of treatment, the highest tolerated dose, the specific formulation used, the route of administration, the response of the patient and the like.

The trAbs employed according to the present invention are preferably administered in an amount of about 5-about 1000 μg, further preferred about 10-about 300 μg, about 10-about 100 μg or about 10-about 50 μg, each per infusion. The optimal amounts may be determined by the skilled artisan by means of experimentation. The number of administrations can be selected by the physician in accordance with the patient's need, particularly the severity of the disease, the patient's response, the dose applied and other various factors known in the art.

Preferably, a blood sample of said individual treated with said trifunctional antibody is provided before or after or, in a further embodiment, before and after having administered said antibody to said individual (patient). Said blood samples are used as a control in order to monitor the development of a secondary humoral immune response against non-EpCAM tumor-associated antigens. In one embodiment, the humoral immune responses are determined in blood serum.

Said antibodies directed against the second tumor-associated cell surface antigen are IgG-antibodies. Preferably, immune responses are monitored which are anti-Her2/neu-IgG, anti-EpCAM IgG, anti-MAGE-A2, anti-MAGE-A3, anti-MAGE-A5, anti-MAGE-AX, anti-NY-ESO-1, anti-NFX2, anti-SSX2, anti-SSX4 anti-Trp2, anti-gp100, anti-tyrosinase, anti-Muc-1*, anti-CEA, anti-telomerase, anti-survivin, anti-CD20, anti-G250, proteoglycans, p53, EGF-R, anti-CA125 anti-MUC, anti-Wue antigen, anti-Lewis Y antigen, anti-HSP-70, anti-HSP-72, anti-GD2, anti-GD3, anti-FAP, anti-Pgp, anti-MCSP, anti-EpHA2, anti-CD33 and anti-cell surface targets GC182, GT468 or GT512 IgG-responses. In one embodiment, said humoral immune response is directed against at least Her2/neu tumor-associated antigen if said trAb was directed against EpCAM. IgG responses might occur against further tumor-associated antigens and might be considered to be monitored. Therefore, the above-described immune responses against tumor-associated antigens have to be understood only as examples and not as limiting the invention.

Said antibodies against the second tumor-associated antigen in said blood sample are preferably induced in an amount of about 1 ng to about 1,000 ng per ml of blood serum. Further ranges of amounts of non-EpCAM antibodies which might be present in the blood serum are 500-1000 ng, 100-500 ng, 10-100 ng, 1-10 ng, 1-500 ng or any other amounts which are covered by the ranges of 1 ng and 1000 ng per ml of blood serum. These amounts may, however, differ depending on the antigen.

The detection of serum antibodies is performed by routine methods as known by a person skilled in the art. Generally the antibodies are detected by forming complexes between the antibody and another antibody able to specifically detect said serum antibodies.

Methods to check the presence of said antibody complexes include, but are not limited to: radioactivity immunoanalysis, ELISA (enzyme-linked immunosorbent assay), sandwich immunoanalysis, and lateral flow immunographic assay. Detection of said complexes involves the use of an antibody labeled directly or indirectly. A usable detection label is described above. The methods are widely known to those skilled in the art. For instance, in case of ELISA, test samples are contacted with a monoclonal antibody specific for said e.g. non-EpCAM serum antibody, or with a Protein A-specific antibody, as coated on microtiter plates, membranes, test strips, and the likes. In one embodiment, microtiter plate wells can be coated with monoclonal antibodies specific for said non-EpCAM serum antibody or with a Protein A-specific antibody, and non-occupied binding sites are blocked with BSA. The coated wells are incubated with test samples, and are examined to see the presence of antigen-antibody complexes. The antibodies can be labeled for detection as described above. Further detection methods available in the art include western-blot, eletrochemiluminescence, detection of serum antibodies by means of target protein chip arrays and any other method which might be appropriate.

It is of further importance that the present inventor has found that said humoral immune responses elicited by trAbs against second tumor-associated antigens like non-EpCAM antigens are induced even in the absence of concurrent chemotherapeutic interventions. Chemotherapeutic drugs usually impair the division of tumor cells, effectively targeting fast-dividing tumor cells. The majority of chemotherapeutic drugs belong to the group of alkylating agents, anti-metabolites, anthracyclines, plant alkaloids, topoisomerase inhibitors and tyrosine kinase inhibitor. Even in cases where the individual in need of a tumor therapy is not treated with said chemotherapeutical agents, said humoral immune response against non-EpCAM antigens develops. The phrase “independent of concurrent chemotherapeutic interventions” has to be understood that the induction of secondary humoral immune responses occurs even in the absence of a chemotherapeutic treatment while said humoral responses occur also when a chemotherapeutic treatment is involved. According to the state of the art, secondary humoral immune responses are induced only in patients undergoing an additional chemotherapy.

It has now been found that the induction of said humoral immune responses against non-EpCAM or non-HER2/neu antigens can be surprisingly correlated with an increased puncture-free survival time of said patient as an improved therapeutic benefit. Said induction of a secondary humoral immune response can, therefore, be used as prospective marker for patients with an increased formation of ascites fluid in order to determine the puncture-free time period. Said humoral immune response against non-EpCAM or non-HER2/neu antigens typically develops about one week after administration of said antibodies. “About one week” involves about 5 to 9, preferably about 7 days. However, said humoral immune response against non-EpCAM or non-HER2/neu antigens can also develop at a later stage and may, therefore, typically be monitored until about 2 to 3 months after administration of said antibodies. Most typically, determination of the induction of a humoral immune response against non-EpCAM or non-HER2/neu antigens is performed up to about 60 days after administration of said antibody.

In a typical case, it has been found that after having administered a trifunctional bispecific antibody directed against the tumor-associated antigen EpCAM, for instance the antibody catumaxomab, an anti-HER2/neu IgG response is induced. However, said induction of an IgG response is not limited to HER2/neu tumor associated antigen but can involve also other tumor-associated antigens which have been already described above. Said non-EpCAM antigens developing in the course of said secondary humoral immune responses are typically present in an amount of at least about 1 ng to about 1,000 ng per ml of blood serum. It is to be understood that the amount of IgG developed against non-EpCAM tumor associated antigens will strongly depend on the time passed after administration of the anti-EpCAM/anti-CD3 directed trifunctional bispecific antibody. With reference to the following example, after about 1 week about 10 to 20 ng of anti-HER2/neu specific IgG antibodies per ml of blood serum have developed, after about 2 to 3 weeks in an amount of about 50 to 70, e.g., about 60 ng per ml of said anti-HER2/neu IgG antibodies have developed and after about 5 weeks about 80 to 120 ng, e.g., about 100 ng of said antibodies per ml of blood serum have developed. Therefore, there is an increase of said development of a humoral immune response depending on the time passed after administration of said trifunctional bispecific intact monoclonal antibody.

The inventors found that inducing humoral immune responses against non-targeted tumor-associated antigens and cancer patients significantly increase the puncture-free survival time of an individual who suffers from an increased formation of ascites fluid. Said puncture-free survival time is generally within a time period of at least about 30 days to about 600 days, preferably in a time period up to 500 days, up to 400 days or at least up to 300 days after administration of the trifunctional bispecific antibody of the present invention.

The prognostic assays described herein, for example, can be used to identify a subject having or not having an improved therapeutic benefit from a treatment involving trifunctional bispecific antibodies as defined herein.

The terms “predicting,” “prognosis,” or grammatical variants thereof, as used herein refer to a prediction of the probable course and outcome of a clinical condition or disease treated by pharmaceutical compositions containing trifunctional bispecific antibodies as defined herein. A prognosis of a patient is usually made by evaluating factors or symptoms of a disease that are indicative of a favorable or unfavorable course or outcome of the disease. The term “prognosis” does not refer to the ability to predict the course or outcome of a condition with 100% accuracy. Instead, the term “prognosis” refers to an increased probability that a certain course or outcome will occur; that is, that a course or outcome is more likely to occur in a patient exhibiting a given condition, when compared to those individuals not exhibiting the condition.

Furthermore, the prognostic assays described herein can be used for designing a personalized therapy for a subject suffering from cancer to determine whether a subject can be administered an agent (e.g., an agonist, antagonist, peptidomimetic, protein, peptide, nucleic acid, small molecule, or other drug candidate) to treat a disease or disorder associated with aberrant expression of tumor-associated antigens. For example, such methods can be used to determine whether a subject can be effectively treated with a trifunctional bispecific antibody or not. Thus, the present invention provides methods for determining whether a subject can be effectively treated with a trifunctional bispecific antibody or not. If not, the patient will be open to any alternative cancer therapies Said alternative cancer therapies involve for example chemotherapy, antibody therapy, radiation therapy, gene therapy etc.

The present invention provides an in vitro method for designing a personalized therapy for a subject suffering from cancer. “Personalized therapy” means that depending on the result of the prognostic test, a particular individualized therapeutic approach is selected in order to provide a treatment for the individual in need of said treatment which is as beneficial as possible. The invention refers to a method comprising the prediction of an improved therapeutic benefit for an individual suffering from a tumor comprising tumor cells expressing a first tumor-associated cell surface antigen, the method comprising:
(a) administering to the individual a therapeutically effective amount of a trifunctional bispecific antibody with the following properties
binding to a T cell via CD3
binding to the first tumor-associated cell surface antigen
binding via its Fc-portion to Fcy-receptor type I, II and/or III positive cells;
(b) determining the level of antibodies against a second tumor-associated cell surface antigen in a blood sample taken from the individual after step (a);
(c) comparing the level of antibodies from step (b) with the level of antibodies against the second tumor-associated antigen in a blood sample taken from the individual prior to step (a);
(d) when an increase in the level of antibodies is detected in step (c), an improved therapeutic benefit is indicated.

In those cases where an increase in the level of antibodies is detected in step (c) with respect to a predetermined value which is to be understood as a reference value, an improved therapeutic benefit is indicated and a specific therapy directed to prevent and/or to treat said cancer is selected, preferably the treatment with the trifunctional bispecific antibodies described herein is continued. In case of evidence that no change, a little increase only or even a decrease in the level of antibodies is detected in step (c), an impaired therapeutic benefit is indicated, and another kind of a more favorable therapeutic approach is to be selected. Said alternative cancer therapies involve for example chemotherapy, therapies with different antibodies as those illustrated herein, radiation therapy, gene therapy etc. These therapies can also be used as additional therapeutic approaches complementing the antibody therapy of the invention.

In those cases where there is an impaired prognosis as no or a small increase in the level of antibodies is detected in step (c), a therapy with one or more of other therapeutically active drugs or therapies known in the art for treating the cancer will be selected. In one example, one or more other anti-proliferative or anticancer therapies are combined with or are administered consecutively with the compounds of the invention. In another example, the compounds disclosed herein are co-administered with one or more of other anticancer drugs known in the art. Anticancer therapies that may be used in combination with the compound of the invention include surgery, radiotherapy (including, but not limited to, gamma-radiation, neutron beam radiotherapy, electron beam radiotherapy, proton therapy, brachytherapy, and systemic radioactive isotopes) and endocrine therapy. Anticancer agents that may be used in combination with the compounds of the invention include biologic response modifiers (including, but not limited to, interferons, interleukins, and tumor necrosis factor (TNF)), hyperthermia and cryotherapy, agents to attenuate any adverse effects (e.g., antiemetics), and other approved chemotherapeutic drugs (e.g., taxol, 5-FU, and analogs thereof).

### Kits

The invention also encompasses kits for performing a method comprising the prediction of an improved therapeutic benefit for an individual suffering from a tumor comprising tumor cells expressing a first tumor-associated cell surface antigen. Such kits can be used to determine if a subject has an improved therapeutic benefit provided there is an increase in the level of antibodies detected in step (c).

In another example, kits can be used to determine the puncture-free time of a subject suffering from ascites cancer which is associated with aberrant expression of a first tumor-associated antigen. In another example, kits can be used for designing a personalized therapy for a subject suffering from cancer which is associated with aberrant expression of a tumor-associated antigen.

The kit, for example, can comprise a labeled compound or agent capable of detecting of the second antibody directed against the second tumor-associated antigen in a biological sample and means for determining the amount of said antibodies in the sample (e.g., an antibody which binds the polypeptide or an oligonucleotide probe which binds to DNA or mRNA encoding the antibody) and means to compare the level of said second antibody before and after treatment with the trifunctional bispecific antibody described herein and directed against a first tumor-associated antigen. Kits can also include instructions for observing if there is an increase in the level of antibodies detected in step (c)of the method of the invention in the tested subject suffering from a cancer associated with aberrant expression of a first tumor-associated antigen.

The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples which can be assayed and compared to the test sample contained. Each component of the kit is usually enclosed within an individual container and all of the various containers are within a single package along with instructions for observing whether the tested subject shows an increase in the level of antibodies to be detected in step (c).

In a further embodiment of the present invention, a method for the induction of secondary humoral immune responses directed against non-targeted second tumor-associated antigens on the surface of tumor cells like EpCAM is provided. An individual suffering from a tumor which is associated with e.g. EpCAM-positive tumor cells is administered a therapeutically effective amount of a trifunctional bispecific antibody binding to a T-cell via CD3, binding to a first tumor-associated antigen like EpCAM and binding via its Fc-portion to Fc-y receptor type I, II and/or III positive cells, thereby inducing humoral immune responses directed against at least one second tumor-associated antigen different from said first tumor-associated antigen like EpCAM. Even in cases where said antibody is administered independently from chemotherapeutical interventions, a humoral immune response is induced. The antibody used is defined in more detail below and is selected from the same pool of antibodies as described with regard to the prognostic method disclosed herein. The amount of antibody used as well as the tumor-associated antigens against which IgG-responses are directed are also the same as described with respect to the method of predicting an improved therapeutic benefit. Specifically, said anti-EpCAM directed trifunctional antibody, preferably catumaxomab, induces an anti-HER2/neu-IgG response. Said induction of humoral immune responses against non-EpCAM antigens in a blood sample is in an amount described above, preferably in an amount of at least about 1 ng per ml of blood serum.

From a therapeutical point of view, the anti-tumor-associated antigen response which develops against non-EpCAM or non_HER2/neu tumor associated antigens might be increased or de novo induced by administering said trifunctional bispecific antibody one, two, three or several times, for instance intraperitoneally or intravenously or subcutaneously. With respect to the method for predicting an improved therapeutic benefit described herein, said secondary antibodies developed against non-EpCAM or non-HER2/neu tumor-associated antigens like HER2/neu or EpCAM can be monitored in the blood serum of cancer patients before and after therapy with said trifunctional antibody in a time interval of about 7 to about 110 days. Said development of non-EpCAM antigens as a secondary immune response is an early biomarker for an improved therapeutic benefit. The higher the amount of secondary humoral immune response directed against targeted or non-targeted tumor-associated antigen is, the better the beneficial therapeutic outcome of a patient suffering from a tumor and treated with said trifunctional bispecific antibodies directed against EpCAM or HER2/neu.

### Antibodies

According to the invention, heterologous intact trifunctional bispecific and/or trispecific antibodies (trAbs) are used in one specific and preferred embodiment of the invention. These antibodies are intact, i.e. have a functional Fc portion, and they must be heterologous in nature, i.e. they must consist of heavy immunoglobulin chains of different subclasses (subclass combinations, also fragments) and/or origin (species).

These intact heterologous trifunctional bispecific and/or trispecific antibodies will be selected to further have the following properties: a) binding to a T cell; b) binding to at least one antigen on a tumor cell; c) binding, by their Fc portion to Fc receptor-positive cells.

Activation of the Fc receptor-positive cell by the trAb is dependent on the subclass or the subclass combination of the antibody heavy chain fragments, respectively. As demonstrated by in vitro experiments, for example, trAbs of the mouse-IgG2a/rat-IgG2b subclass combination are able to bind to, and simultaneously activate, Fc receptor-positive cells leading to an up-regulation or new formation (expression), respectively, of co-stimulatory antigens such as CD40, CD80, or CD86 on the surface of these cells, while bispecific antibodies of the mouse-IgG1/rat-IgG2b subclass combination are able to bind to Fc receptor-positive cells ((1) Haagen et al., J. Immunology, 1995, 154: 1852-1860) but obviously are unable to activate these cells to a comparable extent ((2) Gast et al., Cancer Immunol. Immunother., 1995, 40: 390). Hence, mouse-IgG2a/ratlgG2b isotype combination in the Fc-region of the trAb is particularly preferred. However, this is also true for all other isotype combinations as recited in the present description so that they can be used in other preferred embodiments of the invention.

While the trAbs at the same time bind to and activate the T cell via the binding arms to CD3, co-stimulatory signals derived from the Fc receptor-positive cell bound to the Fc portion of the trAb are transferred to the T cell. I.e. only the combination of T cell activation via one binding arm of the trAb and the concomitant transfer of co-stimulatory signals from the Fc receptor-positive cell to the T cell results in an effective T cell activation.

A further important aspect in the increase of a therapeutical benefit is the possibility of phagocytosis, processing and presentation of tumor components by accessory cells (monocytes, macrophages, dendritic cells, and NK-“natural killer”-cells) which have been directed and activated by the bsab. By this classical mechanism of antigen presentation tumor-specific CD4 cells as well as CD8 positive cells can be generated. Moreover, tumor-specific CD4 cells play an important role in the induction of a humoral immune reaction in the context of the T-B cell cooperation.

Trifunctional bispecific antibodies are able to bind to the T cell receptor complex of the T cell by one binding arm and to tumor-associated antigens by the second binding arm. Thereby, they activate T cells which destroy the tumor cells by releasing cytokines or apoptosis-mediating mechanisms. Furthermore, in the context of their activation by said trifunctional bispecific antibodies it is clearly possible for T cells to recognize tumor-specific antigens via their receptor whereby a long-lasting immunization against the antigen is initiated. In this respect, the intact Fc portion of the trifunctional bispecific antibody is of particular importance mediating the binding to accessory cells such as monocytes, macrophages and dendritic cells and inducing these cells to become themselves cytotoxic and simultaneously transfer important co-stimulatory signals to the T cell. In this manner, a T cell reaction is induced also against antigens the trAb is not directed to.

Redirection of possibly anergized tumor-specific T cells to tumor cells by means of trAbs and concomitant co-stimulation of T cells by accessory cells bound to the Fc portion of the trAbs act to reverse the anergy of cytotoxic T cells (CTLs); i.e. by using intact heterologous trifunctional bispecific antibodies a T cell tolerance existing in the patient against the tumor may be neutralized and, thereby, a long-lasting anti-tumor immunity is induced.

The antibodies used according to the invention are able to reactivate tumor-specific T cells being in an anergic state. Further, they are able to induce e.g. tumor-reactive complement-binding antibodies and thereby a humoral immune reaction.

Binding of the trAbs to the T cell takes place via CD3. Fc receptor-positive cells at least bear an Fc-gamma receptor I, II or III. The Fc-gamma receptor II is preferably of the type Ila or IIb

The trAbs employed according to the invention are able to bind to monocytes, macrophages, dendritic cells, “natural killer” cells (NK cells) and/or activated neutrophils all being Fcgamma receptor I-positive cells.

The antibodies used according to the invention lead to an induction or increase in the expression of CD40, CD80, CD86, ICAM-1, and/or LFA-3 as co-stimulatory antigens and/or cytokine secretion by the Fc receptor-positive cell. The cytokines preferably are IL-1, IL-2, IL-4, IL-6, IL-8, IL-12, and/or TNF-[alpha].

Binding to the T cell takes place via the T cell receptor complex of the T cell.

Preferred are trifunctional antibodies with the following group of isotype combinations in its Fc-region:

rat-IgG2b/mouse-IgG2a,

rat-IgG2b/mouse-IgG2b,

rat-IgG2b/human-IgG1,

mouse[VH-CH1,VL-CL]-human-IgG1/rat-[VH-CH1,VL-CL]-human-IgG1-[hinge]-human-IgG3*-[CH2-CH3]

wherein*=caucasian allotypes G3m(b+g)=no binding to protein A.

Preferably said trifunctional bispecific antibody has the isotype combination rat-IgG2b/mouse-IgG2a.

In one embodiment, the invention uses the trifunctional bispecific antibody catumaxomab which is already known in the art. Reference is made for instance to. Shen et al., Curr. Opin. Mol. Ther. 10:273-284 (2008); Chelius et al., mAbs 2:309-319 (2010); Linke et al., mAbs 2:129-136 (2010) Catumaxomab is an anti EpCAM, anti-CD3 directed trifunctional bispecific antibody with the isotype (subclass) combination rat-IgG2b/mouse-IgG2a.

The preparation of monoclonal antibodies preferably originating from mammals, e.g. methods, as for example described in Köhler and Milstein (Nature 256 (1975), 495), in Harlow and Lane (Antibodies, A Laboratory Manual (1988), Cold Spring Harbour) or in Galfré (Meth. Enzymol. 73 (1981), 3). Furthermore, it is possible to prepare the antibodies described by means of recombinant DNA technology according to techniques obvious to the skilled artisan (see Kurucz et al., J. Immunol. 154 (1995), 4576; Hollinger et al., Proc. Natl. Acad. Sc. USA go (1993), 6444). The antibodies used in the present method can be designed and manufactured by a person skilled in the art without undue burden; e.g. Greenwood et al. disclose the exchange of single immunoglobulin domains (for instance CH2) by suitable cloning technique. By using these cloning technique antibody combinations like mouse-[VH-CH1,VL-CL]-human-IgG1/rat-[VH-CH1,VL-CL]-human-IgG1-[hinge]-human-IgG3*-[CH2-CH3] wherein *=caucasian allotypes G3m(b+g)=no binding to protein A.

On the one hand, the preparation of trifunctional bispecific antibodies may be performed using recombinant DNA technology or by hybrid-hydridoma fusion technique (see for example Milstein et al., Nature 305 (1983), 537). By this technique, hybridoma cell lines producing antibodies each having one of the desired specificities are fused, and recombinant cell lines producing antibodies with both specificities are identified and isolated.

For the sake of complete disclosure, reference is made for instance to U.S. Pat. No. 6,994,853 which is incorporated herein by reference completely.

According to the invention, there are for example used intact trifunctional bispecific antibodies. TrAbs are a combination of two antibody semi-molecules (each of one H and L immunoglobulin chain) each representing one specificity and having in addition an Fc portion which performs the well-known effector functions. Preferably, they are prepared by quadroma technology. This method of preparation is described representatively in DE-A-44 19 399. This document is incorporated by reference in its entirety for the purpose of complete disclosure also with respect to a definition of trifunctional bispecific antibodies.

Methods of producing polyclonal and monoclonal antibodies are known to those of ordinary skill in the art, and many antibodies are available. See, e.g., Coligan, Current Protocols in Immunology Wiley/Greene, N.Y., 1991; and Harlow and Lane, Antibodies: A Laboratory Guide Manual, Cold Spring Harbor Press, NY, 1989; Stites et al., (eds.) Basic and Clinical Immunology (4th ed.) Lange Medical Publications, Los Altos, Calif., and references cited therein; Goding, Monoclonal Antibodies: Principles and Practice (2nd ed.) Academic Press, New York, N.Y.] 986; and Köhler and Milstein, Nature 256: 495-497, 1975. Other suitable techniques for antibody preparation include selection of libraries of recombinant antibodies in phage or similar vectors. See, Huse et al., Science 246: 1275-1281, 1989; and Ward et al., Nature 341: 544-546, 1989. “Specific” monoclonal and polyclonal antibodies and antisera (or antiserum) will usually bind with a KD of at least about 0.1 μM, optionally at least about 0.01 μM or 0.001 μM, or even better.

Detailed methods for preparation of chimeric (humanized) antibodies can be found in U.S. Pat. No. 5,482,856. Additional details on humanization and other antibody production and engineering techniques can be found in Borrebaeck (ed), Antibody Engineering, 2″<d> Edition Freeman and Company, NY, 1995; McCafferty et al., Antibody Engineering, A Practical Approach, IRL at Oxford Press, Oxford, England, 1996, and Paul Antibody Engineering Protocols Humana Press, Towata, N.J., 1995.

Also other methods of preparation may be employed as long as they result in the intact trifunctional bispecific antibodies defined above.

The present invention is now described also with regard to a non-limiting example.

### EXAMPLE 1

Induction of Anti-Herz/Neu Humoral Immunity by Catumaxomab

In a phase IIb study CASIMAS catumaxomab was investigated with/without prednisolone premedication in patients with MA due to epithelial cancer. Briefly, patients received four 3-hour infusions of 10, 20, 50 and 150 μg catumaxomab over 11 days. Plasma samples of 23 patients were quantified for autologous anti-EpCAM and anti-HER2/neu immunoglobulin responses by ELISA.

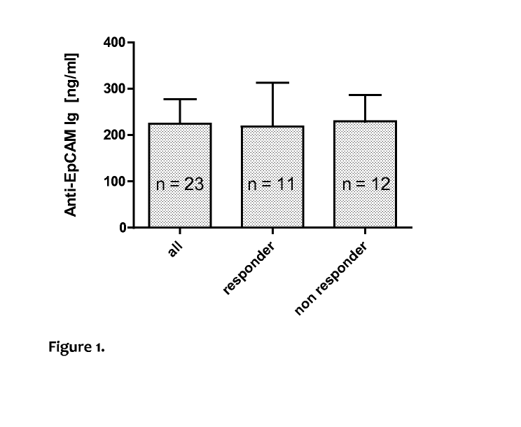

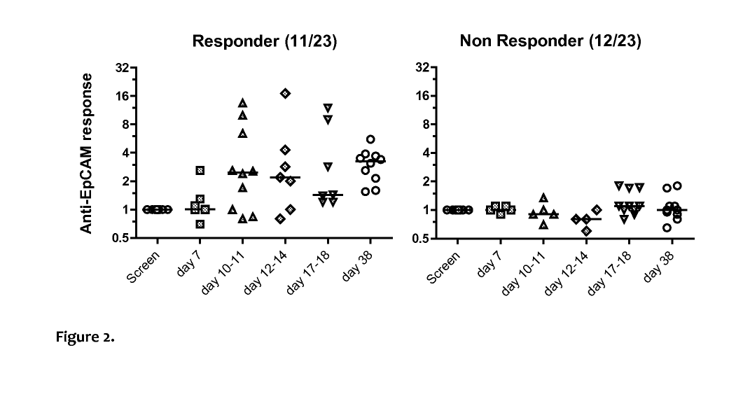

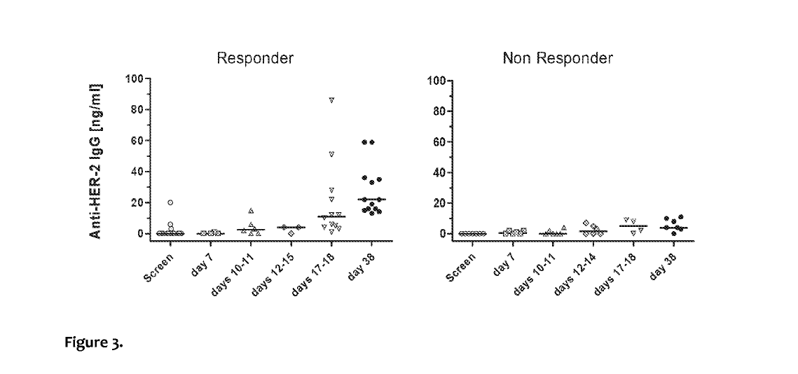

At screening EpCAM-positive tumor cells were detected in the ascites fluid of all 23 patients. Thereof, 11 individuals (48%) showed a median 3-4 times increase of the initial anti-EpCAM immunoglobulin titer after treatment, whereas in 12/23 patients (52%) the titer did not increase (FIG. 1). Although anti-EpCAM immunoglobulin screening values varied largely (2-855 ng/ml) no immunoglobulin significant distinction between the anti-EpCAM responder group and non-responders was measured (p=0.35; Mann Whitney test) (FIG. 2).

Moreover, in 14/25 patients (56%) HER2/neu-specific immunoglobulin could be quantified. In contrast to the augmented anti-EpCAM antibody levels which rapidly appeared (day 10) anti-HER2/neu responses developed de novo, started delayed (day 18) and peaked at the last sample collection (day 38, median=22 ng/ml) (FIG. 2). The results indicate an active immunization induced by catumaxomab. Interestingly, the humoral immune responses were not restricted to the EpCAM targeted but antigen spreading to the non-targeted HER2/neu tumor-associated antigen occurred.

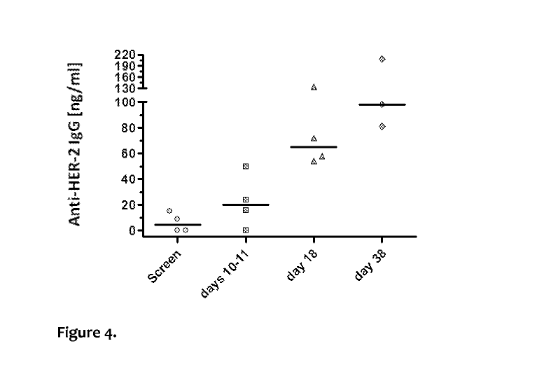

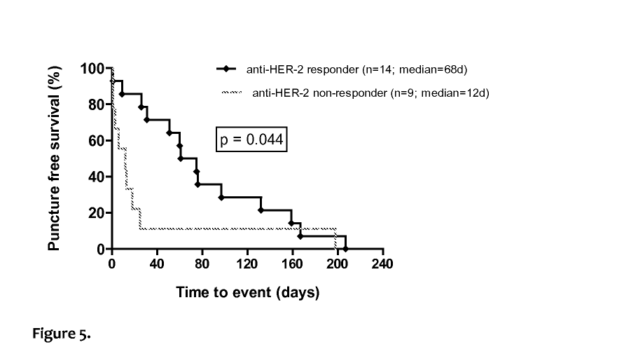

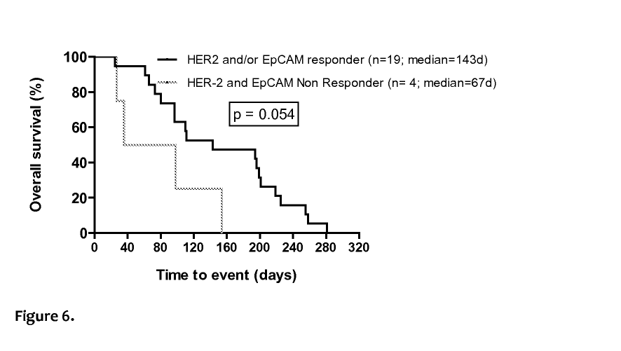

Moreover, MA patients of the CASIMAS study who did benefit from the catumaxomab therapy, but finally relapsed more than 60 days later, received a second catumaxomab treatment cycle (four 3-hour infusions of 10, 20, 50 and 150 μg catumaxomab over 11 days). Of note, 4 of 5 these MA patients demonstrated an anti-HER2/neu immunoglobulin booster reaction that was stronger and developed faster as compared to MA patients who were treated only with a single catumaxomab treatment cycle (median values at day 18: 65 vs. 11 ng/ml) (FIG. 4). Most importantly, the humoral anti-HER2/neu responses positively correlated with the clinical outcome (median of PFS of anti-HER2/neu responders versus non-responders, 68 days vs. 12 days, log rank test p=0.044) (FIG. 5). In addition, there was also a positive trend towards improved overall survival (OS) for patients showing either anti-HER2/neu and/or anti-EpCAM antibody responses (n=19) in comparison with patients who did not develop any responses to these particular antigens (FIG. 6).

### EXAMPLE 2

Along a treatment schedule (phase I/II ERTUSO trial) for cancer patients suffering from HER2/neu (1+/SISH- or FISH-positive, 2+ and 3+) expressing solid tumors with the bispecific trifunctional antibody ertumaxomab (anti-HER2/neu×anti-CD3) secondary immune responses raised against meaningful tumor-associated antigens e.g. the non-targeted EpCAM and targeted HER2/neu are analyzed. By means of interferon-γ-specific EliSpot analysis with HLA-A2 restricted peptide epitopes of relevant antigens like EpCAM or HER2/neu and appropriate EpCAM- or HER2/neu-specific ELISA assays are performed to characterize secondary cell-mediated or humoral immune responses, respectively. Of note, ertumaxomab is administered intravenously once weekly in two cycles, each cycle lasting 28 days with a 21 day treatment-free interval in-between the cycles. Ertumaxomab administration during the two treatment cycles will follow a predefined dose escalation scheme, consisting of 5 ascending doses per cycle with each infusion lasting 3 hours. Furthermore, the ertumaxomab dose levels will be identical in the first and second cycle. The end of study is planned for day 108 post initial screening and subsequent treatment start.

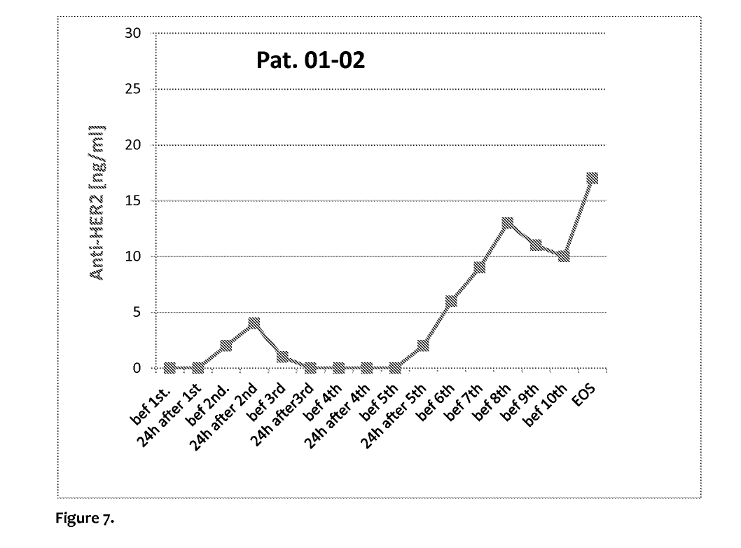

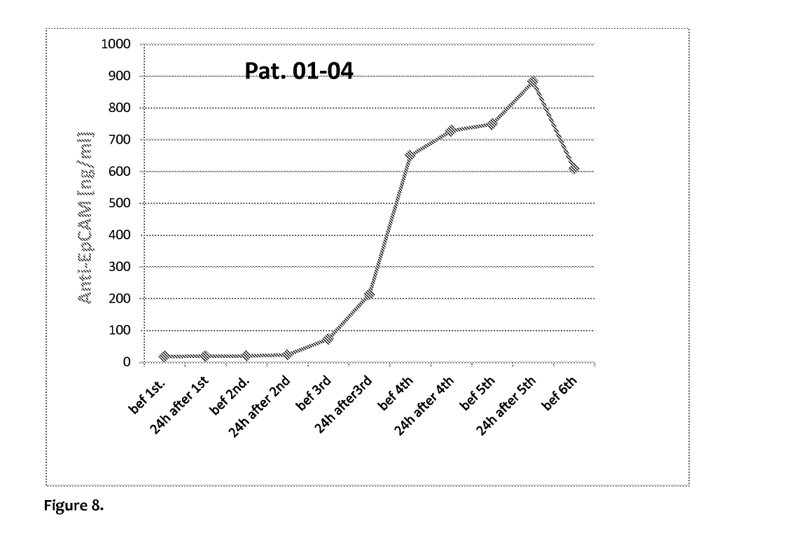

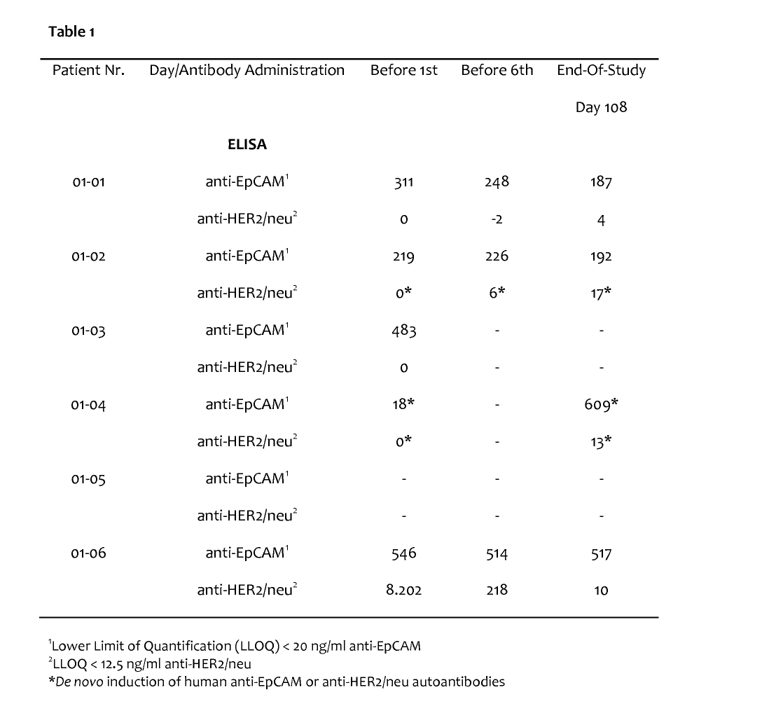

It is expected that in accordance with the catumaxomab-mediated induction of secondary immune responses against non-targeted antigens (in this case HER2/neu as already decribed here) the Her2/neu-positive cancer patients treated with ertumaxomab can be divided into responders and non-responders along the treatment schedule up to end-of-study day 108. Moreover, the responder population can be further subdivided into two groups that show (i) pre-existing but augmented and (ii) non-pre-existing de novo induced immune responses i.e. human autoantibodies directed against EpCAM and/or HER2/neu and/or HLA-A2 restricted T cell responses towards EpCAM or HER2/neu for instance by end-of-study day 108. After the enrollment of six cancer patients (Table 1) the first immunological analysis of serum samples from two patients (01-02 and 01-04) in fact showed the de novo induction of human anti-HER2/neu autoantibodies by end-of-study day 10 (FIG. 7). Furthermore, this analysis also showed that the serum samples of one cancer patient (01-04) contained de novo induced and non-targeted human anti-EpCAM autoantibodies indicating antigen spreading (Table 1, FIG. 8).

The present invention is not to be limited in scope by the specific embodiments described therein. Indeed, various modifications of the inventions in addition to those described herein, will become apparent to those skilled in the art from the foregoing description and accompanying figures. Such modifications are intended to fall within the scope of the present claims.

Those skilled in the art will recognize or be able to ascertain using no more than routine experimentation, many equivalents to the specific embodiments of the invention described herein. Such equivalents are intended to be encompassed by the following claims.

All references cited herein are incorporated by reference in their entirety and for all purposes to the same extend as if each individual publication or patent or patent application was specifically and individually indicated to be incorporated by reference in its entirety for all purposes.

1. A method for predicting an improved therapeutic benefit for an individual suffering from a tumor comprising tumor cells expressing a first tumor-associated cell surface antigen, the method comprising:
(a) administering to the individual a therapeutically effective amount of a trifunctional bispecific antibody with the following properties
binding to a T cell via CD3
binding to the first tumor-associated cell surface antigen
binding via its Fc-portion to Fcy-receptor type I, II and/or III positive cells;
(b) determining the level of antibodies against a second tumor-associated cell surface antigen in a blood sample taken from the individual after step (a);
(c) comparing the level of antibodies from step (b) with the level of antibodies against the second tumor-associated antigen in a blood sample taken from the individual prior to step (a);
(d) when an increase in the level of antibodies is detected in step (c), an improved therapeutic benefit is indicated.

2. The method according to claim 1, wherein said trifunctional antibody is a rat/mouse bispecific antibody.

3. The method according to claim 1, wherein said trifunctional antibody is selected from at least one member of antibodies with one of the following isotype combinations in its Fc-region:
rat-IgG2b/mouse-IgG2a,
rat-IgG2b/mouse-IgG2b,
rat-IgG2b/human-IgG1,
mouse-[VH-CH1,VL-CL]-human-IgG1/rat-[VH-CH1,VL-CL]-human-IgG1-[hinge]-human-IgG3*-[CH2-CH3]
wherein *=caucasian allotypes G3m(b+g)=no binding to protein A.

4. The method according to claim 1, wherein said level of antibodies against a second tumor-associated cell surface antigen in a blood sample taken from the individual after step (a) is determined between about 7 to about 60 days after having administered said bispecific antibody.

5. The method according to claim 1, wherein said first tumor-associated antigen is selected from the group consisting of EpCAM, Her2/neu, MAGE-A2, MAGE-A3, MAGE-A5, MAGE-AX, NY-ESO-1, NFX2, SSX2, SSX4 Trp2, gp100, tyrosinase, Muc-1*, CEA, telomerase, survivin, CD20, G250, proteoglycans, p53, EGF-R, CA125 MUC, Wue antigen, Lewis Y antigen, HSP-27, HSP-70, HSP-72, HSP-90, GD2, GD3, FAP, Pgp, MCSP, EpHA2, CD33 and cell surface targets GC182, GT468 or GT512.

6. The method according to claim 1, wherein said second tumor-associated antigen is different from the first tumor-associated antigen and is selected from the group consisting of EpCAM, Her2/neu, MAGE-A2, MAGE-A3, MAGE-A5, MAGE-AX, NY-ESO-1, NFX2, SSX2, SSX4 Trp2, gp100, tyrosinase, Muc-1*, CEA, telomerase, survivin, CD20, G250, proteoglycans, p53, EGF-R, CA125 MUC, Wue antigen, Lewis Y antigen, HSP-27, HSP-70, HSP-72, HSP-90, GD2, GD3, FAP, Pgp, MCSP, EpHA2, CD33 and cell surface targets GC182, GT468 or GT512.

7. The method according to claim 1, wherein said first tumor-associated antigen is EpCAM and said second tumor-associated antigen is at least Her2/neu or wherein said first tumor-associated antigen is Her2/neu and said second tumor-associated antigen is at least EpCAM.

8-12. (canceled)

13. A method for the induction of secondary humoral immune responses directed against non-targeted tumor-associated antigens in an individual suffering from a tumor carrying a first tumor-associated antigen on the surface of tumor cells comprising
administering to an individual in need thereof a therapeutically effective amount of a trifunctional bispecific antibody with the following properties
binding to a T cell via CD3
binding to the first tumor-associated antigen
binding via its Fc-portion to Fcy-receptor type I, II and/or III positive cells
inducing in said individual a humoral immune response directed against at least one second tumor-associated antigen different from said first tumor-associated antigen by said administration of a trifunctional bispecific antibody, wherein said induction of a humoral immune response is independent from concurrent chemotherapeutic interventions.

14. The method according to claim 13, wherein said trifunctional antibody is a rat/mouse bispecific antibody.

15. The method according to claim 13, wherein said trifunctional antibody is selected from at least one member of antibodies with one of the following group of isotype combinations in its Fc-region:
rat-IgG2b/mouse-IgG2a,
rat-IgG2b/mouse-IgG2b,
rat-IgG2b/human-IgG1,
mouse-[VH-CH1,VL-CL]-human-IgG1/rat-[VH-CH1,VL-CL]-human-IgG1-[hinge]-human-IgG3*-[CH2-CH3]
wherein *=caucasian allotypes G3m(b+g)=no binding to protein A.

16. The method according to claim 13, wherein said humoral immune response is directed against at least one of the tumor-associated antigens Her2/neu, EpCAM, MAGE-A2, MAGE-A3, MAGE-A5, MAGE-AX, NY-ESO-1, NFX2, SSX2, SSX4, Trp2, gp100, tyrosinase, Muc-1*, CEA, telomerase, survivin, CD20, G250, proteoglycans, p53, EGF-R, CA125, MUC, Wue antigen, Lewis Y antigen, HSP-27, HSP-70, HSP-72, HSP-90, GD2, GD3, FAP, Pgp, MCSP, EpHA2, CD33 and cell surface targets GC182, GT468 and GT512.

17-18. (canceled)

19. The method according to claim 13, wherein said induction of a humoral immune response against the second antigens in said blood sample is in an amount of at least about lng/ml of blood serum.

20-21. (canceled)

22. The method according to claim 13 wherein said second tumor-associated antigen is different from the first tumor-associated antigen and is selected from the group consisting of EpCAM, Her2/neu, MAGE-A2, MAGE-A3, MAGE-A5, MAGE-AX, NY-ESO-1, NFX2, SSX2, SSX4 Trp2, gp100, tyrosinase, Muc-1*, CEA, telomerase, survivin, CD20, G250, proteoglycans, p53, EGF-R, CA125 MUC, Wue antigen, Lewis Y antigen, HSP-27, HSP-70, HSP-72, HSP-90, GD2, GD3, FAP, Pgp, MCSP, EpHA2, CD33 and cell surface targets GC182, GT468 or GT512.

23. (canceled)

24. A method for the induction of secondary humoral immune responses directed against non-targeted second tumor-associated antigens in an individual suffering from a tumor carrying a first tumor-associated antigen on the surface of tumor cells comprising
administering to an individual in need thereof a therapeutically effective amount of a trifunctional bispecific antibody with the following properties
binding to a T cell via CD3
binding to the first tumor-associated antigen
binding via its Fc-portion to Fcg-receptor type I, II and/or III positive cells,
inducing in said individual an augmented or de novo humoral immune response directed against at least one second tumor-associated antigen different from said first tumor-associated antigen by said administration of a trifunctional bispecific antibody, wherein said induction of a humoral immune response is independent from concurrent chemotherapeutic interventions, and wherein the amount of said antibody induced against said second or further tumor-associated antigen after administration of said therapeutically effective amount of a trifunctional bispecific antibody is compared with the amount of said second or further antibody before administration of said therapeutically effective amount of a trifunctional bispecific antibody wherein an increase in the amount of said antibody induced against said second or further tumor-associated antigen indicates an improved therapeutic benefit.

25. The method of claim 24, wherein said first tumor-associated antigen is EpCAM and wherein said second tumor-associated antigen is Her2/neu, or wherein said first tumor-associated antigen is Her2/neu and wherein said second tumor-associated antigen is EpCAM.

26. A method for treating a patient suffering from a tumor expressing multiple tumor-associated antigens, the method comprising administering to the patient a therapeutically effective amount of a trifunctional antibody with the following properties:
binding to a T cell via CD3;
binding to a tumor-associated antigen; and
binding via its Fc-portion to Fcg-receptor type I, II and/or III positive cells,
wherein the patient has been previously identified by the method of claim 1 to have an improved therapeutic benefit.
